(19) United States
(12) Patent Application Publication
(10) Pub. No.: US 2009/0149544 A1
(43) Pub. Date: Jun. 11, 2009

(54) Alpha-aminoamide derivatives

(75) Inventor: Julie F. Liu, Lexington, MA (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: CoNCERT Pharmaceuticals, Inc., Lexington, MA (US)

(21) Appl. No.: 12/288,723

(22) Filed: Oct. 22, 2008

Related U.S. Application Data
(60) Provisional application No. 60/981,588, filed on Oct. 22, 2007.

(51) Int. Cl. C07C 237/20; A61K 31/165; C12N 5/06; A61P 25/00; A61P 25/28; A61P 25/30; A61P 25/24

(52) U.S. Cl. 514/620; 564/165; 435/375

(57) ABSTRACT

This invention relates to novel alpha-aminoamide derivatives, their pharmaceutically acceptable salts, solvates, and hydrates thereof. This invention also provides compositions comprising a compound of this invention and the use of such compositions in methods of treating diseases and conditions that are beneficially treated by administering an inhibitor of monoamine oxidase type B (MAO-B) and/or a sodium (Na⁺) channel blocker, and/or a calcium (Ca²⁺) channel modulator.

### RELATED APPLICATION

This application claims the benefit of U.S. Provisional Application No. 60/981,588, filed on Oct. 22, 2007. The entire teachings of the above application are incorporated herein by reference.

### BACKGROUND OF THE INVENTION

Safinamide, also known as (S)-2-(4-(3-fluorobenzyloxy)benzyl-amino) propionamide methanesulfonate, combines multiple mechanisms of action, including reversible inhibition of monoamine oxidase type B (MAO-B), sodium (Na⁺) channel blocking activity, calcium (Ca²⁺) channel modulation, dopamine reuptake inhibition, and glutamate level modulation. Inhibition of MAO-B reduces the metabolic inactivation of dopamine in patients, while the Na⁺ channel blockade selectively affects those neurons with abnormal firing patterns and leaves normal activity unaltered.

Safinamide is currently in phase III clinical trials for Parkinson's disease and phase II for restless legs syndrome.

Possible metabolites of safinamide include alaninamide and 4-(3-fluorobenzyloxy)benzaldehyde. Strolin, B et al., Prog Brain Res, 1995, 106:123-34.

Adverse events typical of anti-epileptics and MAO-B inhibitors include headache, somnolence and lightheadedness. Patients dosed with safinamide experienced few of these side effects in phase I clinical trials and no notable side effects related to the drug were observed in phase II and III trials. Stocchi, F et al., Neurology, 2004, 63(4):746-748.

Despite the beneficial activities of safinamide, there is a continuing need for new compounds to treat the aforementioned diseases and conditions.

### SUMMARY OF THE INVENTION

This invention relates to novel alpha-aminoamide derivatives or pharmaceutically acceptable salts thereof. This invention also provides compositions comprising a compound of this invention and the use of such compositions in methods of treating diseases and conditions that are beneficially treated by administering an inhibitor of monoamine oxidase type B (MAO-B) and/or a sodium (Na⁺) channel blocker, and/or a calcium (Ca²⁺) channel modulator.

### DETAILED DESCRIPTION OF THE INVENTION

The terms “ameliorate” and “treat” are used interchangeably and include both therapeutic and prophylactic treatment. Both terms mean decrease, suppress, attenuate, diminish, arrest, or stabilize the development or progression of a disease (e.g., a disease or disorder delineated herein), lessen the severity of the disease or improve the symptoms associated with the disease.

“Disease” means any condition or disorder that damages or interferes with the normal function of a cell, tissue, or organ.

It will be recognized that some variation of natural isotopic abundance occurs in a synthesized compound depending upon the origin of chemical materials used in the synthesis. Thus, a preparation of safinamide will inherently contain small amounts of deuterated isotopologues. The concentration of naturally abundant stable hydrogen and carbon isotopes, notwithstanding this variation, is small and immaterial as compared to the degree of stable isotopic substitution of compounds of this invention. See, for instance, Wada E et al., Seikagaku 1994, 66:15; Ganes L Z et al., Comp Biochem Physiol Mol Integr Physiol 1998, 119:725. In a compound of this invention, when a particular position is designated as having deuterium, it is understood that the abundance of deuterium at that position is substantially greater than the natural abundance of deuterium, which is 0.015%. A position designated as having deuterium typically has a minimum isotopic enrichment factor of at least 3340 (50.1% deuterium incorporation) at each atom designated as deuterium in said compound.

The term “isotopic enrichment factor” as used herein means the ratio between the isotopic abundance and the natural abundance of a specified isotope.

In other embodiments, a compound of this invention has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

In the compounds of this invention any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition. Also unless otherwise stated, when a position is designated specifically as “D” or “deuterium”, the position is understood to have deuterium at an abundance that is at least 3340 times greater than the natural abundance of deuterium, which is 0.015% (i.e., at least 50.1% incorporation of deuterium).

The term “isotopologue” refers to a species that differs from a specific compound of this invention only in the isotopic composition thereof.

The term “compound,” when referring to a compound of this invention, refers to a collection of molecules having an identical chemical structure, except that there may be isotopic variation among the constituent atoms of the molecules. Thus, it will be clear to those of skill in the art that a compound represented by a particular chemical structure containing indicated deuterium atoms, will also contain lesser amounts of isotopologues having hydrogen atoms at one or more of the designated deuterium positions in that structure. The relative amount of such isotopologues in a compound of this invention will depend upon a number of factors including the isotopic purity of deuterated reagents used to make the compound and the efficiency of incorporation of deuterium in the various synthesis steps used to prepare the compound. However, as set forth above the relative amount of such isotopologues in toto will be less than 49.9% of the compound. In other embodiments, the relative amount of such isotopologues in toto will be less than 47.5%, less than 40%, less than 32.5%, less than 25%, less than 17.5%, less than 10%, less than 5%, less than 3%, less than 1%, or less than 0.5% of the compound.

The invention also provides salts, solvates or hydrates of the compounds of the invention.

A salt of a compound of this invention is formed between an acid and a basic group of the compound, such as an amino functional group, or a base and an acidic group of the compound, such as a carboxyl functional group. According to another embodiment, the compound is a pharmaceutically acceptable acid addition salt.

The term “pharmaceutically acceptable,” as used herein, refers to a component that is, within the scope of sound medical judgment, suitable for use in contact with the tissues of humans and other mammals without undue toxicity, irritation, allergic response and the like, and are commensurate with a reasonable benefit/risk ratio. A “pharmaceutically acceptable salt” means any non-toxic salt that, upon administration to a recipient, is capable of providing, either directly or indirectly, a compound of this invention. A “pharmaceutically acceptable counterion” is an ionic portion of a salt that is not toxic when released from the salt upon administration to a recipient.

Acids commonly employed to form pharmaceutically acceptable salts include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephathalate, sulfonate, xylene sulfonate, phenylacetate, phenylpropionate, phenylbutyrate, citrate, lactate, β-hydroxybutyrate, glycolate, maleate, tartrate, methanesulfonate, propanesulfonate, naphthalene-1-sulfonate, naphthalene-2-sulfonate, mandelate and other salts. In one embodiment, pharmaceutically acceptable acid addition salts include those formed with mineral acids such as hydrochloric acid and hydrobromic acid, and especially those formed with organic acids such as maleic acid.

As used herein, the term “hydrate” means a compound which further includes a stoichiometric or non-stoichiometric amount of water bound by non-covalent intermolecular forces.

As used herein, the term “solvate” means a compound which further includes a stoichiometric or non-stoichiometric amount of solvent such as water, acetone, ethanol, methanol, dichloromethane, 2-propanol, or the like, bound by non-covalent intermolecular forces.

The compounds of the present invention (e.g., compounds of Formula I or A), may contain an asymmetric carbon atom, for example, as the result of deuterium substitution or otherwise. As such, compounds of this invention can exist as either individual enantiomers, or mixtures of the two enantiomers. Accordingly, a compound of the present invention may exist as either a racemic mixture or a scalemic mixture, or as individual respective stereoisomers that are substantially free from another possible stereoisomer. The term “substantially free of other stereoisomers” as used herein means less than 25% of other stereoisomers, preferably less than 10% of other stereoisomers, more preferably less than 5% of other stereoisomers and most preferably less than 2% of other stereoisomers, or less than “X”% of other stereoisomers (wherein X is a number between 0 and 100, inclusive) are present. Methods of obtaining or synthesizing an individual enantiomer for a given compound are known in the art and may be applied as practicable to final compounds or to starting material or intermediates.

Unless otherwise indicated, when a disclosed compound is named or depicted by a structure without specifying the stereochemistry and has one or more chiral centers, it is understood to represent all possible stereoisomers of the compound.

The term “stable compounds,” as used herein, refers to compounds which possess stability sufficient to allow for their manufacture and which maintain the integrity of the compound for a sufficient period of time to be useful for the purposes detailed herein (e.g., formulation into therapeutic products, intermediates for use in production of therapeutic compounds, isolatable or storable intermediate compounds, treating a disease or condition responsive to therapeutic agents).

“D” and “d” both refer to deuterium. “Stereoisomer” refers to both enantiomers and diastereomers. “Tert”, “^{t}” and “t-” each refer to tertiary. “US” refers to the United States of America.

Throughout this specification, a variable may be referred to generally (e.g., “each R”) or may be referred to specifically (e.g., R¹, R², R³, etc.). Unless otherwise indicated, when a variable is referred to generally, it is meant to include all specific embodiments of that particular variable.

### Therapeutic Compounds

The present invention provides a compound of Formula A:

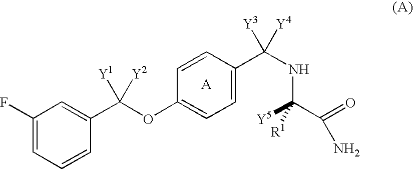

or a pharmaceutically acceptable salt thereof, wherein:

Ring A contains 0-4 deuterium atoms;

each Y is independently selected from hydrogen and deuterium;

R¹ is selected from —CH₃, —CH₂D, —CHD₂ and —CD₃; and

when R¹ is —CH₃ and Ring A contains 0 deuterium atoms, then at least one Y is deuterium.

Other embodiments of a compound of Formula A include those wherein:

a) Y¹ and Y² are the same;

b) Y³ and Y⁴ are the same;

c) Ring A is substituted with 0 or 4 deuterium atoms; or

d) R¹ is selected from —CH₃ and —CD₃.

More specific embodiments of a compound of Formula A include those having properties set forth in two or more of a) through d), above. For example, a and b, a and c, a and d, b and c, b and d, c and d, a and b and c, a and b and d, a and c and d, b and c and d, and a and b and c and d.

In another set of embodiments, any atom not designated as deuterium in any of the embodiments set forth above is present at its natural isotopic abundance.

One embodiment of the present invention provides a compound of Formula I:

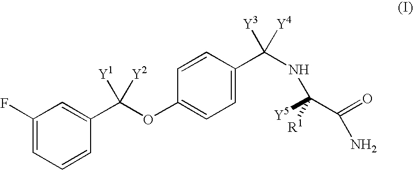

or a pharmaceutically acceptable salt, solvate, or hydrate thereof, wherein:

each Y is independently selected from hydrogen and deuterium;

R¹ is selected from —CH₃, —CH₂D, —CHD₂ and —CD₃; and

when R¹ is —CH₃, then at least one Y is deuterium.

In this embodiment, each hydrogen attached to one of the phenyl rings is present at its natural abundance.

Other embodiments of a compound of Formula I include those wherein:

a) Y¹ and Y² are the same;

b) Y³ and Y⁴ are the same; or

c) R¹ is selected from —CH₃ and —CD₃.

More specific embodiments of a compound of Formula I include those having properties set forth in two or more of a) through c), above. For example, a and b, a and c, b and c, and a and b and c.

In yet another embodiment, the compound is selected from any one of the following compounds, where any atom not designated as deuterium is present at its natural abundance:

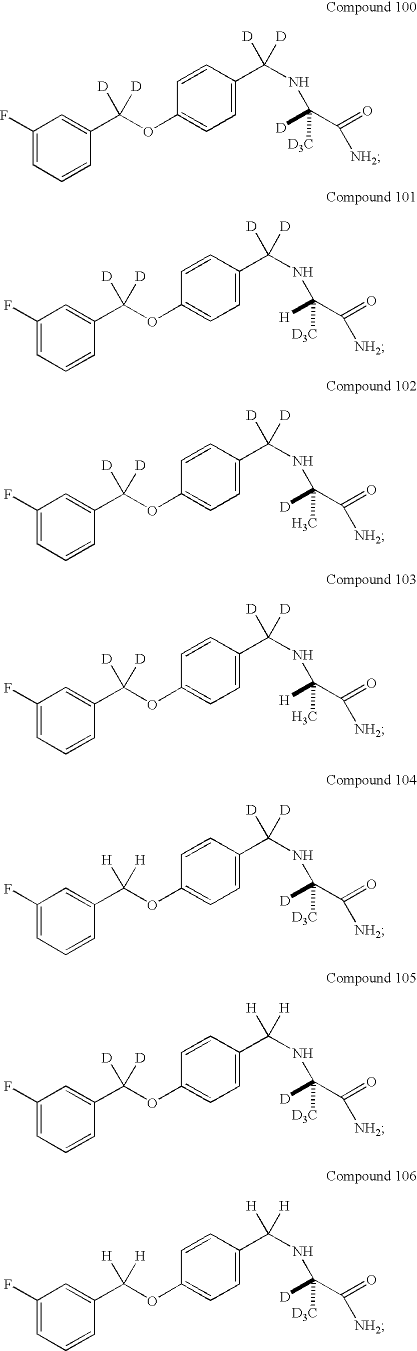

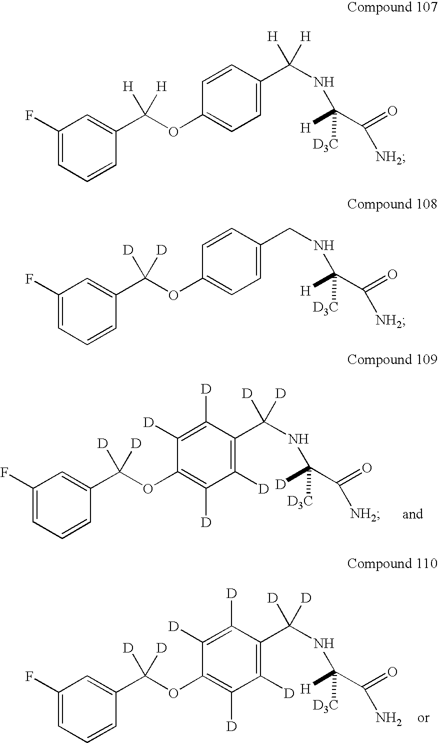

a pharmaceutically acceptable salt of any of the foregoing.

In another set of embodiments, any atom not designated as deuterium in any of the embodiments set forth above is present at its natural isotopic abundance.

### Exemplary Synthesis

The synthesis of compounds of Formula I or A can be readily achieved by synthetic chemists of ordinary skill. Such methods can be carried out utilizing corresponding deuterated and optionally, other isotope-containing reagents and/or intermediates to synthesize the compounds delineated herein, or invoking standard synthetic protocols known in the art for introducing isotopic atoms to a chemical structure. Relevant procedures and intermediates are disclosed, for instance in Sorbera, L A et al., Drugs Fut, 2001, 26(8):745; Pevarello, P et al., J Med Chem, 1998, 41(4):579; and Varasi, M et al., 12th Int Symp Med Chem, (September 13-17, Basel) 1992, Abst P-089.C. The schemes below illustrate how compounds of Formula I or A may be prepared.

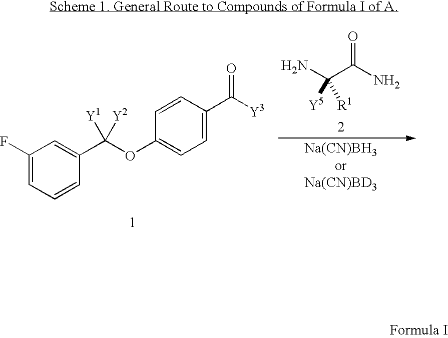

Scheme 1 shows a general route for preparing compounds of Formula I or A. As described generally in the literature cited above, reductive alkylation of appropriately-deuterated aldehyde 1 with appropriately-deuterated aminoamide 2 using either sodium cyanoborohydride or sodium cyanoborodeuteride affords compounds of Formula I or A.

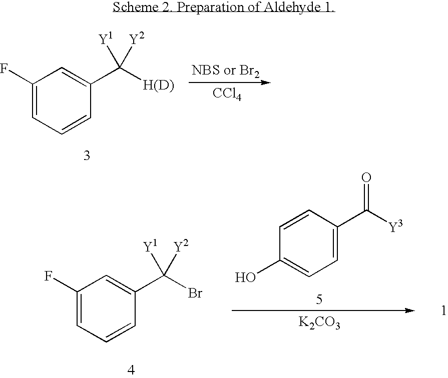

Scheme 2 depicts a route toward the preparation of aldehydes 1, which are useful starting materials for Scheme 1. Appropriately-deuterated toluene derivative 3 is brominated to provide bromide 4 using either NBS (see Li, H et al., Wuji Huaxue Xuebao, 2006, 22(7):1231-1234 or bromine (see Kuliev, A M et al., Katalit. Prevrashcheniya Organ. Soedin. Baku, 1981. 51-5, from: Ref. Zh., Khim. 1983, Abstr. No. 1Zh415). Alkylation of appropriately-deuterated 4-hydroxybenzaldehyde 5 with bromide 4 in the presence of potassium carbonate provides aldehydes 1 following the general method of Dawson, M I et al., Journal of Medicinal Chemistry, 2007, 50(11):2622-2639.

For example, commercially-available 3-fluoro(methyl-d₃)-benzene,

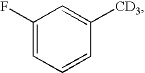

may be used as starting material 3 in Scheme 2 to ultimately produce compounds of Formula I wherein Y¹ and Y² are deuterium.

Alternatively, commercially-available methyl 3-fluorobenzoate may be reduced with LiAlH₄ (or LiAlD₄) to afford 3-fluorobenzenemethanol (or 3-fluorobenzenemethan-d2-ol), which may be treated with PBr₃ to provide 4 wherein Y¹ and Y² are both hydrogen (or Y¹ and Y² are both deuterium.)

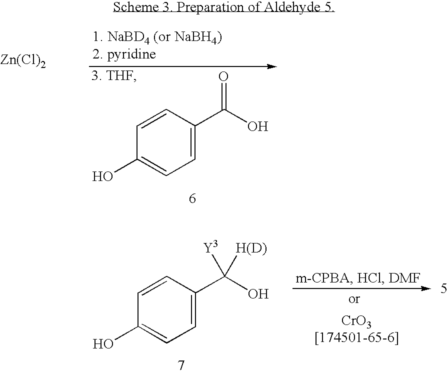

Scheme 3 illustrates a route toward the preparation of aldehydes 5, which are useful reagents for Scheme 2. According to the general method of Zeynizadeh, B et al., Journal of Chemical Research, Synopses, 2003, 8:522-525, zinc chloride is treated with either sodium borohydride or sodium borodeuteride, followed by pyridine and then by 4-hydroxybenzoic acid 6 to yield alcohol 7. Oxidation of the alcohol with m-CPBA according to the procedure of Kim, H R et al., Synthetic Communications, 1990, 20(5):637-40, affords aldehyde 5. Alternatively, alcohol 7 may be oxidized with chromium trioxide in the ionic liquid 1-butyl-3-methylimidazolium tetrafluoroborate [174501-65-6] to provide aldehydes 5 following the procedure of Zheng, Y-F et al., Journal of Chemical Research, 2005, 12:753-754.

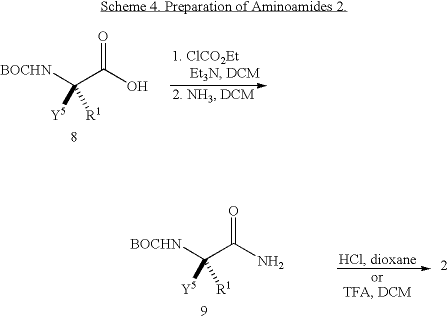

Scheme 4 shows a route toward the preparation of aminoamides 2, which are useful reagents for Scheme 1. Appropriately-deuterated N—BOC-L-alanine is treated with ethyl chloroformate in dichloromethane (DCM) followed by ammonia to afford amide 9 following the procedure of Alvarado, C et al., Tetrahedron Letters, 2007, 48(4):603-607. Deprotection of the amine with either HCl or TFA provides aminoamides 2.

For example, commercially-available L-alanine-2,3,3,3-d₄-N-t-BOC,

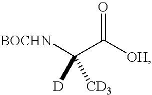

may be used as starting material 8 in Scheme 4 to ultimately produce compounds of Formula I wherein Y⁵ is deuterium and R¹ is CD₃.

In another example, commercially-available L-alanine-3,3,3-d₃-N-t-BOC,

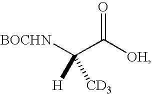

may be used as starting material 8 in Scheme 4 to ultimately produce compounds of Formula I wherein Y⁵ is hydrogen and R¹ is CD₃.

The specific approaches and compounds shown above are not intended to be limiting. The chemical structures in the schemes herein depict variables that are hereby defined commensurately with chemical group definitions (moieties, atoms, etc.) of the corresponding position in the compound formulae herein, whether identified by the same variable name (i.e., Y¹, Y², Y³, etc.) or not. The suitability of a chemical group in a compound structure for use in the synthesis of another compound is within the knowledge of one of ordinary skill in the art. Additional methods of synthesizing compounds of Formula I or A and their synthetic precursors, including those within routes not explicitly shown in schemes herein, are within the means of chemists of ordinary skill in the art. Methods for optimizing reaction conditions and, if necessary, minimizing competing by-products, are known in the art. In addition to the synthetic references cited herein, reaction schemes and protocols may be determined by the skilled artisan by use of commercially available structure-searchable database software, for instance, SciFinder® (CAS division of the American Chemical Society), STN® (CAS division of the American Chemical Society), CrossFire Beilstein® (Elsevier MDL), or internet search engines such as Google® or keyword databases such as the US Patent and Trademark Office text database.

The methods described herein may also additionally include steps, either before or after the steps described specifically herein, to add or remove suitable protecting groups in order to ultimately allow synthesis of the compounds herein. In addition, various synthetic steps may be performed in an alternate sequence or order to give the desired compounds. Synthetic chemistry transformations and protecting group methodologies (protection and deprotection) useful in synthesizing the applicable compounds are known in the art and include, for example, those described in Larock R, Comprehensive Organic Transformations, VCH Publishers (1989); Greene T W et al., Protective Groups in Organic Synthesis, 3^{rd} Ed., John Wiley and Sons (1999); Fieser L et al., Fieser and Fieser's Reagents for Organic Synthesis, John Wiley and Sons (1994); and Paquette L, ed., Encyclopedia of Reagents for Organic Synthesis, John Wiley and Sons (1995) and subsequent editions thereof.

Combinations of substituents and variables envisioned by this invention are only those that result in the formation of stable compounds.

### Compositions

The invention also provides pyrogen-free pharmaceutical compositions comprising an effective amount of a compound of Formula I or A (e.g., including any of the formulae herein), or a pharmaceutically acceptable salt thereof; and a pharmaceutically acceptable carrier. The carrier(s) are “acceptable” in the sense of being compatible with the other ingredients of the formulation and, in the case of a pharmaceutically acceptable carrier, not deleterious to the recipient thereof in an amount used in the medicament.

Pharmaceutically acceptable carriers, adjuvants and vehicles that may be used in the pharmaceutical compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

If required, the solubility and bioavailability of the compounds of the present invention in pharmaceutical compositions may be enhanced by methods well-known in the art. One method includes the use of lipid excipients in the formulation. See “Oral Lipid-Based Formulations: Enhancing the Bioavailability of Poorly Water-Soluble Drugs (Drugs and the Pharmaceutical Sciences),” David J. Hauss, ed. Informa Healthcare, 2007; and “Role of Lipid Excipients in Modifying Oral and Parenteral Drug Delivery: Basic Principles and Biological Examples,” Kishor M. Wasan, ed. Wiley-Interscience, 2006.

Another known method of enhancing bioavailability is the use of an amorphous form of a compound of this invention optionally formulated with a poloxamer, such as LUTROL™ and PLURONIC™ (BASF Corporation), or block copolymers of ethylene oxide and propylene oxide. See U.S. Pat. No. 7,014,866; and United States patent publications 20060094744 and 20060079502.

The pharmaceutical compositions of the invention include those suitable for oral, rectal, nasal, topical (including buccal and sublingual), vaginal or parenteral (including subcutaneous, intramuscular, intravenous and intradermal) administration. In certain embodiments, the compound of the formulae herein is administered transdermally (e.g., using a transdermal patch or iontophoretic techniques). Other formulations may conveniently be presented in unit dosage form, e.g., tablets, sustained release capsules, and in liposomes, and may be prepared by any methods well known in the art of pharmacy. See, for example, Remington's Pharmaceutical Sciences, Mack Publishing Company, Philadelphia, Pa. (17th ed. 1985).

Such preparative methods include the step of bringing into association with the molecule to be administered ingredients such as the carrier that constitutes one or more accessory ingredients. In general, the compositions are prepared by uniformly and intimately bringing into association the active ingredients with liquid carriers, liposomes or finely divided solid carriers, or both, and then, if necessary, shaping the product.

In certain embodiments, the compound is administered orally. Compositions of the present invention suitable for oral administration may be presented as discrete units such as capsules, sachets, or tablets each containing a predetermined amount of the active ingredient; a powder or granules; a solution or a suspension in an aqueous liquid or a non-aqueous liquid; an oil-in-water liquid emulsion; a water-in-oil liquid emulsion; packed in liposomes; or as a bolus, etc. Soft gelatin capsules can be useful for containing such suspensions, which may beneficially increase the rate of compound absorption.

In the case of tablets for oral use, carriers that are commonly used include lactose and corn starch. Lubricating agents, such as magnesium stearate, are also typically added. For oral administration in a capsule form, useful diluents include lactose and dried cornstarch. When aqueous suspensions are administered orally, the active ingredient is combined with emulsifying and suspending agents. If desired, certain sweetening and/or flavoring and/or coloring agents may be added.

Compositions suitable for oral administration include lozenges comprising the ingredients in a flavored basis, usually sucrose and acacia or tragacanth; and pastilles comprising the active ingredient in an inert basis such as gelatin and glycerin, or sucrose and acacia.

Compositions suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze dried (lyophilized) condition requiring only the addition of the sterile liquid carrier, for example water for injections, immediately prior to use. Extemporaneous injection solutions and suspensions may be prepared from sterile powders, granules and tablets.

Such injection solutions may be in the form, for example, of a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant.

The pharmaceutical compositions of this invention may be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax and polyethylene glycols.

The pharmaceutical compositions of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art. See, e.g.: Rabinowitz J D and Zaffaroni A C, U.S. Pat. No. 6,803,031, assigned to Alexza Molecular Delivery Corporation.

Topical administration of the pharmaceutical compositions of this invention is especially useful when the desired treatment involves areas or organs readily accessible by topical application. For topical application topically to the skin, the pharmaceutical composition should be formulated with a suitable ointment containing the active components suspended or dissolved in a carrier. Carriers for topical administration of the compounds of this invention include, but are not limited to, mineral oil, liquid petroleum, white petroleum, propylene glycol, polyoxyethylene polyoxypropylene compound, emulsifying wax, and water. Alternatively, the pharmaceutical composition can be formulated with a suitable lotion or cream containing the active compound suspended or dissolved in a carrier. Suitable carriers include, but are not limited to, mineral oil, sorbitan monostearate, polysorbate 60, cetyl esters wax, cetearyl alcohol, 2-octyldodecanol, benzyl alcohol, and water. The pharmaceutical compositions of this invention may also be topically applied to the lower intestinal tract by rectal suppository formulation or in a suitable enema formulation. Topically-transdermal patches and iontophoretic administration are also included in this invention.

Application of the subject therapeutics may be local, so as to be administered at the site of interest. Various techniques can be used for providing the subject compositions at the site of interest, such as injection, use of catheters, trocars, projectiles, pluronic gel, stents, sustained drug release polymers or other device which provides for internal access.

Thus, according to yet another embodiment, the compounds of this invention may be incorporated into compositions for coating an implantable medical device, such as prostheses, artificial valves, vascular grafts, stents, or catheters. Suitable coatings and the general preparation of coated implantable devices are known in the art and are exemplified in U.S. Pat. Nos. 6,099,562; 5,886,026; and 5,304,121. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluorosilicone, polysaccharides, polyethylene glycol, phospholipids or combinations thereof to impart controlled release characteristics in the composition. Coatings for invasive devices are to be included within the definition of pharmaceutically acceptable carrier, adjuvant or vehicle, as those terms are used herein.

According to another embodiment, the invention provides a method of coating an implantable medical device comprising the step of contacting said device with the coating composition described above. It will be obvious to those skilled in the art that the coating of the device will occur prior to implantation into a mammal.

According to another embodiment, the invention provides a method of impregnating an implantable drug release device comprising the step of contacting said drug release device with a compound or composition of this invention. Implantable drug release devices include, but are not limited to, biodegradable polymer capsules or bullets, non-degradable, diffusible polymer capsules and biodegradable polymer wafers.

According to another embodiment, the invention provides an implantable medical device coated with a compound or a composition comprising a compound of this invention, such that said compound is therapeutically active.

According to another embodiment, the invention provides an implantable drug release device impregnated with or containing a compound or a composition comprising a compound of this invention, such that said compound is released from said device and is therapeutically active.

Where an organ or tissue is accessible because of removal from the patient, such organ or tissue may be bathed in a medium containing a composition of this invention, a composition of this invention may be painted onto the organ, or a composition of this invention may be applied in any other convenient way.

In another embodiment, a composition of this invention further comprises a second therapeutic agent. The second therapeutic agent may be selected from any compound or therapeutic agent known to have or that demonstrates advantageous properties when administered with a compound having the same mechanism of action as safinamide. Such agents include those indicated as being useful in combination with safinamide, including but not limited to, those described in WO 2004089353.

Preferably, the second therapeutic agent is an agent useful in the treatment or prevention of a disease or condition selected from Parkinson's disease, restless legs syndrome, addictive disorders, head pain conditions such as migraine, cluster headache, or other severe headache, chronic and neuropathic pain, epilepsy, neuroprotection, depression, and diseases treated with an antispastic and/or hypnotic agent.

In one embodiment, the invention provides separate dosage forms of a compound of this invention and one or more of any second therapeutic agents, wherein the compound and second therapeutic agent are associated with one another. The term “associated with one another” as used herein means that the separate dosage forms are packaged together or otherwise attached to one another such that it is readily apparent that the separate dosage forms are intended to be sold and administered together (within less than 24 hours of one another, consecutively or simultaneously).

In the pharmaceutical compositions of the invention, the compound of the present invention is present in an effective amount. As used herein, the term “effective amount” refers to an amount which, when administered in a proper dosing regimen, is sufficient to treat (therapeutically or prophylactically) the target disorder. For example, and effective amount is sufficient to reduce or ameliorate the severity, duration or progression of the disorder being treated, prevent the advancement of the disorder being treated, cause the regression of the disorder being treated, or enhance or improve the prophylactic or therapeutic effect(s) of another therapy.

The interrelationship of dosages for animals and humans (based on milligrams per meter squared of body surface) is described in Freireich et al., (1966) Cancer Chemother. Rep 50: 219. Body surface area may be approximately determined from height and weight of the patient. See, e.g., Scientific Tables, Geigy Pharmaceuticals, Ardsley, N.Y., 1970, 537.

In one embodiment, an effective amount of a compound of this invention can range from about 0.4 mg to about 3000 mg per treatment. In more specific embodiments the range is from about 4 to 1500 mg, or from about 8 to 600 mg, or most specifically from about 40 to 300 mg per treatment. Treatment typically is administered once daily.

Effective doses will also vary, as recognized by those skilled in the art, depending on the diseases treated, the severity of the disease, the route of administration, the sex, age and general health condition of the patient, excipient usage, the possibility of co-usage with other therapeutic treatments such as use of other agents and the judgment of the treating physician. For example, guidance for selecting an effective dose can be determined by reference to the prescribing information for safinamide.

For pharmaceutical compositions that comprise a second therapeutic agent, an effective amount of the second therapeutic agent is between about 20% and 100% of the dosage normally utilized in a monotherapy regime using just that agent. Preferably, an effective amount is between about 70% and 100% of the normal monotherapeutic dose. The normal monotherapeutic dosages of these second therapeutic agents are well known in the art. See, e.g., Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), each of which references are incorporated herein by reference in their entirety.

It is expected that some of the second therapeutic agents referenced above will act synergistically with the compounds of this invention. When this occurs, it will allow the effective dosage of the second therapeutic agent and/or the compound of this invention to be reduced from that required in a monotherapy. This has the advantage of minimizing toxic side effects of either the second therapeutic agent of a compound of this invention, synergistic improvements in efficacy, improved ease of administration or use and/or reduced overall expense of compound preparation or formulation.

### Methods of Treatment

In another embodiment, the invention provides a method of modulating MAO-B, Na⁺ channel, and/or Ca²⁺ channel activity in a cell, comprising contacting a cell with one or more compounds of Formula I or A herein.

According to another embodiment, the invention provides a method of treating a a disease that is beneficially treated by safinamide in a patient in need thereof comprising the step of administering to said patient an effective amount of a compound or a composition of this invention. Such diseases are well known in the art and are disclosed in, but not limited to the following patents and published applications: WO 1990014334, WO 1999035125, WO 2004062655, WO 2004089353, and WO 2005102300. Such diseases include, but are not limited to, Parkinson's disease, restless legs syndrome, addictive disorders, head pain conditions such as migraine, cluster headache, or other severe headache, chronic and neuropathic pain, epilepsy, and depression.

In one particular embodiment, the method of this invention is used to treat a disease or condition selected from Parkinson's disease, and restless legs syndrome in a patient in need thereof.

Methods delineated herein also include those wherein the patient is identified as in need of a particular stated treatment. Identifying a patient in need of such treatment can be in the judgment of a patient or a health care professional and can be subjective (e.g. opinion) or objective (e.g. measurable by a test or diagnostic method).

In another embodiment, any of the above methods of treatment comprises the further step of co-administering to said patient one or more second therapeutic agents. The choice of second therapeutic agent may be made from any second therapeutic agent known to be useful for co-administration with safinamide. The choice of second therapeutic agent is also dependent upon the particular disease or condition to be treated.

The term “co-administered” as used herein means that the second therapeutic agent may be administered together with a compound of this invention as part of a single dosage form (such as a composition of this invention comprising a compound of the invention and an second therapeutic agent) or as separate, multiple dosage forms. Alternatively, the additional agent may be administered prior to, consecutively with, or following the administration of a compound of this invention. In such combination therapy treatment, both the compounds of this invention and the second therapeutic agent(s) are administered by conventional methods. The administration of a composition of this invention, comprising both a compound of the invention and a second therapeutic agent, to a patient does not preclude the separate administration of that same therapeutic agent, any other second therapeutic agent or any compound of this invention to said patient at another time during a course of treatment.

Effective amounts of these second therapeutic agents are well known to those skilled in the art and guidance for dosing may be found in patents and published patent applications referenced herein, as well as in Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), and other medical texts. However, it is well within the skilled artisan's purview to determine the second therapeutic agent's optimal effective-amount range.

In one embodiment of the invention, where a second therapeutic agent is administered to a subject, the effective amount of the compound of this invention is less than its effective amount would be where the second therapeutic agent is not administered. In another embodiment, the effective amount of the second therapeutic agent is less than its effective amount would be where the compound of this invention is not administered. In this way, undesired side effects associated with high doses of either agent may be minimized. Other potential advantages (including without limitation improved dosing regimens and/or reduced drug cost) will be apparent to those of skill in the art.

In yet another aspect, the invention provides the use of a compound of Formula I or A alone or together with one or more of a second therapeutic agent in the manufacture of a medicament, either as a single composition or as separate dosage forms, for treatment or prevention in a patient of a disease, disorder or symptom set forth above. Another aspect of the invention is a compound of Formula I or A for use in the treatment or prevention in a patient of a disease, disorder or symptom thereof delineated herein.

### Diagnostic Methods and Kits

The compounds and compositions of this invention are also useful as reagents in methods for determining the concentration of safinamide in solution or biological sample such as plasma, examining the metabolism of safinamide and other analytical studies.

According to one embodiment, the invention provides a method of determining the concentration, in a solution or a biological sample, of safinamide, comprising the steps of:
a) adding a known concentration of a compound of Formula I or A to the solution of biological sample;
b) subjecting the solution or biological sample to a measuring device that distinguishes safinamide from a compound of Formula I or A;
c) calibrating the measuring device to correlate the detected quantity of the compound of Formula I or A with the known concentration of the compound of Formula I or A added to the biological sample or solution; and
d) measuring the quantity of safinamide in the biological sample with said calibrated measuring device; and
e) determining the concentration of safinamide in the solution of sample using the correlation between detected quantity and concentration obtained for a compound of Formula I or A.

Measuring devices that can distinguish safinamide from the corresponding compound of Formula I or A include any measuring device that can distinguish between two compounds that differ from one another only in isotopic abundance. Exemplary measuring devices include a mass spectrometer, NMR spectrometer, or IR spectrometer.

In another embodiment, the invention provides a method of evaluating the metabolic stability of a compound of Formula I or A comprising the steps of contacting the compound of Formula I or A with a metabolizing enzyme source for a period of time and comparing the amount of the compound of Formula I or A with the metabolic products of the compound of Formula I or A after the period of time.

In a related embodiment, the invention provides a method of evaluating the metabolic stability of a compound of Formula I or A in a patient following administration of the compound of Formula I or A. This method comprises the steps of obtaining a serum, urine or feces sample from the patient at a period of time following the administration of the compound of Formula I or A to the subject; and comparing the amount of the compound of Formula I or A with the metabolic products of the compound of Formula I or A in the serum, urine or feces sample.

The present invention also provides kits for use to treat Parkinson's disease and restless legs syndrome. These kits comprise (a) a pharmaceutical composition comprising a compound of Formula I or A or a salt, hydrate, or solvate thereof, wherein said pharmaceutical composition is in a container; and (b) instructions describing a method of using the pharmaceutical composition to treat Parkinson's disease and restless legs syndrome.

The container may be any vessel or other sealed or sealable apparatus that can hold said pharmaceutical composition. Examples include bottles, ampules, divided or multi-chambered holders bottles, wherein each division or chamber comprises a single dose of said composition, a divided foil packet wherein each division comprises a single dose of said composition, or a dispenser that dispenses single doses of said composition. The container can be in any conventional shape or form as known in the art which is made of a pharmaceutically acceptable material, for example a paper or cardboard box, a glass or plastic bottle or jar, a re-sealable bag (for example, to hold a “refill” of tablets for placement into a different container), or a blister pack with individual doses for pressing out of the pack according to a therapeutic schedule. The container employed can depend on the exact dosage form involved, for example a conventional cardboard box would not generally be used to hold a liquid suspension. It is feasible that more than one container can be used together in a single package to market a single dosage form. For example, tablets may be contained in a bottle, which is in turn contained within a box. In one embodiment, the container is a blister pack.

The kits of this invention may also comprise a device to administer or to measure out a unit dose of the pharmaceutical composition. Such device may include an inhaler if said composition is an inhalable composition; a syringe and needle if said composition is an injectable composition; a syringe, spoon, pump, or a vessel with or without volume markings if said composition is an oral liquid composition; or any other measuring or delivery device appropriate to the dosage formulation of the composition present in the kit.

In certain embodiment, the kits of this invention may comprise in a separate vessel of container a pharmaceutical composition comprising a second therapeutic agent, such as one of those listed above for use for co-administration with a compound of this invention.

### Evaluation of Metabolic Stability

Certain in vitro liver metabolism studies have been described previously in the following references, each of which is incorporated herein in their entirety: Obach, R S, Drug Metab Disp, 1999, 27:1350; Houston, J B et al., Drug Metab Rev, 1997, 29:891; Houston, J B, Biochem Pharmacol, 1994, 47:1469; Iwatsubo, T et al., Pharmacol Ther, 1997, 73:147; and Lave, T, et al., Pharm Res, 1997, 14:152.

Microsomal Assay. Human liver microsomes (20 mg/mL) are obtained from Xenotech, LLC (Lenexa, Kans.). β-nicotinamide adenine dinucleotide phosphate, reduced form (NADPH), magnesium chloride (MgCl₂), and dimethyl sulfoxide (DMSO) are purchased from Sigma-Aldrich. The incubation mixtures are prepared according to the Table:

TABLE
Reaction Mixture Composition for Human Liver Microsome Study

| | | |
|---|---|---|
| Liver Microsomes | 3.0 | mg/mL |
| Potassium Phosphate, pH 7.4 | 100 | mM |
| Magnesium Chloride | 10 | mM |

Determination of Metabolic Stability: Two aliquots of this reaction mixture are used for a compound of this invention. The aliquots are incubated in a shaking water bath at 37° C. for 3 minutes. The test compound is then added into each aliquot at a final concentration of 0.5 μM. The reaction is initiated by the addition of cofactor (NADPH) into one aliquot (the other aliquot lacking NADPH serves as the negative control). Both aliquots are then incubated in a shaking water bath at 37° C. Fifty microliters (50 μL) of the incubation mixtures are withdrawn in triplicate from each aliquot at 0, 5, 10, 20, and 30 minutes and combined with 50 μL of ice-cold acetonitrile to terminate the reaction. The same procedure is followed for safinamide and the positive control. Testing is done in triplicate.

Data analysis: The in vitro half lives (t_{1/2}s) for test compounds are calculated from the slopes of the linear regression of % parent remaining (ln) vs incubation time relationship using the following equation:

in vitro t_{1/2}=0.693/k

k=−[slope of linear regression of % parent remaining (ln) vs incubation time]

Data analysis is performed using Microsoft Excel Software.

The metabolic stability of compounds of Formula I or A is tested using pooled liver microsomal incubations. Full scan LC-MS analysis is then performed to detect major metabolites. Samples of the test compounds, exposed to pooled human liver microsomes, are analyzed using HPLC-MS (or MS/MS) detection. For determining metabolic stability, multiple reaction monitoring (MRM) is used to measure the disappearance of the test compounds. For metabolite detection, QI full scans are used as survey scans to detect the major metabolites.

SUPERSOMES™ Assay. Various human cytochrome P450-specific SUPERSOMES™ are purchased from Gentest (Woburn, Mass., USA). A 1.0 mL reaction mixture containing 25 pmole of SUPERSOMES™, 2.0 mM NADPH, 3.0 mM MgCl, and 1 μM of a compound of Formula I or A in 100 mM potassium phosphate buffer (pH 7.4) is incubated at 37° C. in triplicate. Positive controls contain 1 μM of safinamide instead of a compound of Formula I or A. Negative controls used Control Insect Cell Cytosol (insect cell microsomes that lacked any human metabolic enzyme) purchased from GenTest (Woburn, Mass., USA). Aliquots (50 μL) are removed from each sample and placed in wells of a multi-well plate at various time points (e.g., 0, 2, 5, 7, 12, 20, and 30 minutes) and to each aliquot is added 50 μL of ice cold acetonitrile with 3 μM haloperidol as an internal standard to stop the reaction.

Plates containing the removed aliquots are placed in −20° C. freezer for 15 minutes to cool. After cooling, 100 μL of deionized water is added to all wells in the plate. Plates are then spun in the centrifuge for 10 minutes at 3000 rpm. A portion of the supernatant (100 μL) is then removed, placed in a new plate and analyzed using Mass Spectrometry.

### EXAMPLES

### Example 1

Synthesis of (S)-2-amino-2,3,3,3-d₄-propanamide (12). Intermediate 12 was prepared as outlined in Scheme 5 below. Details of the synthesis are set forth below.

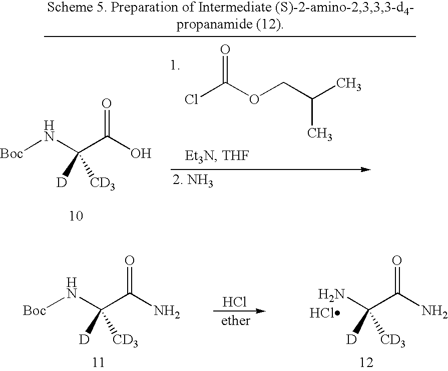

Synthesis of (S)-tert-butyl 1-amino-1-oxo-2,3,3,3-d₄-propan-2-ylcarbamate (11). To a solution of 10 (CDN, 98 atom % D, 1.00 g, 5.2 mmol) in THF (30 mL) was added triethylamine (1.43 mL, 10.4 mmol, 2 equiv) with stirring. The resulting mixture was cooled to −50° C. and isobutylchloroformate (1.25 mL, 10.4 mmol, 2 equiv) was added dropwise. The resulting mixture was stirred for 2 hours (h) at room temperature (rt) before NH₃ gas was bubbled into it over 30 minutes (min). The mixture was then poured into CH₂Cl₂ (50 mL) and the resultant precipitate was removed by filtration. The filtrate was washed with 15% aqueous NaHCO₃, dried over Na₂SO₄, and concentrated in vacuo. The residue was dried by lyophilization to yield 11 as a white solid which was used directly in the next step.

Synthesis of (S)-2-amino-2,3,3,3-d₄-propanamide (12). To a solution of 11 in MeOH (5 mL) was added 1 M HCl in ether (30 mL) with stirring. The mixture was stirred for 1 h at rt and then was concentrated in vacuo to yield 524 mg (78% over 2 steps) of the desired product 12 as a white solid.

### Example 2

Synthesis of (S)-2-amino-3,3,3-d₃-propanamide (15). Intermediate 15 was prepared as outlined in Scheme 6 below. Details of the synthesis are set forth below.

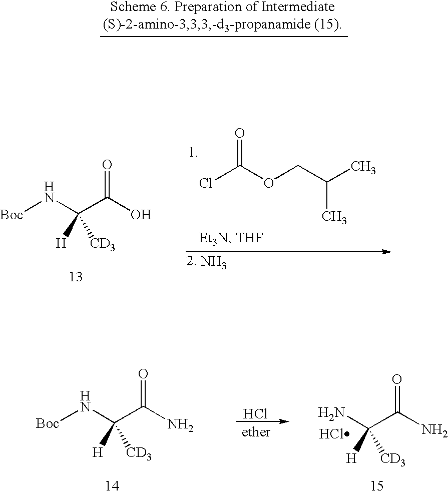

Synthesis of (S)-tert-butyl 1-amino-1-oxo-3,3,3-d₃-propan-2-ylcarbamate (14). To a solution of 13 (CDN, 99 atom % D, 500 mg, 2.6 mmol) in THF (30 mL) was added triethylamine (72 mL, 5.2 mmol, 2 equiv) with stirring. The mixture was cooled to −50° C. and isobutylchloroformate (67 mL, 5.2 mmol, 2 equiv) was added dropwise. The resulting mixture was stirred for 2 h at rt before NH₃ gas was bubbled into it over 30 min. The mixture was then poured into CH₂Cl₂ (50 mL), and the resultant precipitate was removed by filtration. The filtrate was washed with 15% aqueous NaHCO₃, dried over Na₂SO₄, and concentrated in vacuo. The residue was dried by lyophilization to yield 325 mg (65%) of 14 as a white solid.

Synthesis of (S)-2-amino-3,3,3-d₃-propanamide (15). To a solution of 14 (325 mg, 1.7 mmol) in MeOH (5 mL) was added 1 M HCl in ether (30 mL) with stirring. The mixture was stirred for 1 h at rt and then was concentrated in vacuo to yield 125 mg (58%) of the desired product 15 as a white solid.

### Example 3

Synthesis of 4-hydroxy-2,3,5,6-d₄-benz(aldehyde-d₁) (18). Intermediate 18 was prepared as outlined in Scheme 7 below. Details of the synthesis are set forth below.

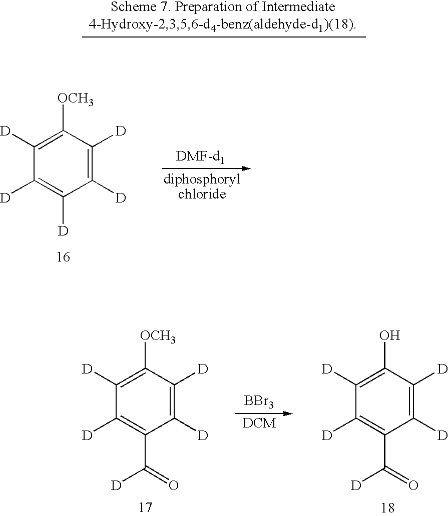

Synthesis of 4-methoxy-2,3,5,6-d₄-benz(aldehyde-d₁) (17). A stirring solution of anisole 16 (2.00 g, 17.7 mmol) in DMF-d₁ (2.05 mL, 26.5 mmol, 1.5 equiv) under an atmosphere of N₂ was cooled to 0° C., and diphosphoryl chloride (3.67 mL, 26.5 mmol, 1.5 equiv) was added dropwise over 10 min. The resulting mixture was heated to 105° C., stirred for 24 h, and then was allowed to cool to rt. The cooled mixture was poured onto ice (25 g), and the pH was adjusted to 10 with 5N NaOH at 0° C., and then extracted with CH₂Cl₂ (4×25 mL). The combined organic layers were dried over Na₂SO₄, filtered, and concentrated under reduced pressure to a yellow oil which was further dried under high vacuum overnight to yield 963 mg (38.5%) of desired intermediate 17. HPLC (method: 150 mm C18-RP column-gradient method 5-95% ACN; Wavelength: 280 nm): retention time: 3.87 min. MS (M+H): 142.2.

Synthesis of 4-Hydroxy-2,3,5,6-d₄-benz(aldehyde-d₁) (18). To a well-stirred solution of 17 (963 mg, 6.8 mmol) in CH₂Cl₂ (20 mL) was added BBr₃ (381 μl, 7.2 mmol, 1.05 equiv). The reaction mixture was stirred for 30 min at rt and then was quenched with MeOH at 0° C., concentrated under reduced pressure, dissolved in MeOH, and concentrated under reduced pressure again. The resultant crude material was purified by automated flash column chromatography (25-30% EtOAc/heptane), followed by crystallization from heptane (5 mL)/EtOAc (2 mL) to yield 569 mg (66%) of desired product 18. HPLC (method: 150 mm C18-RP column-gradient method 5-95% ACN; Wavelength: 280 nm): retention time: 2.95 min; 98% purity. MS (M+H): 128.1.

### Example 4

Synthesis of (S)-2-(4-((3-fluorophenyl)methoxy-d₂)benzylamino)-(3,3,3-d₃-propan)amide (108). Compound 108 was prepared as outlined in Scheme 8 below. Details of the synthesis are set forth below.

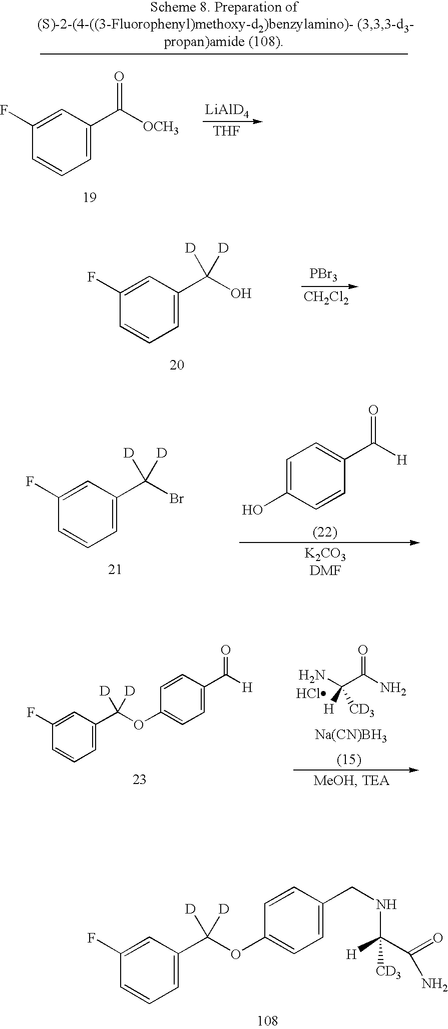

Synthesis of (3-Fluorophenyl)methanol-d₂ (20). A solution of ester 19 (2.34 mL, 26.0 mmol) in THF (6.4 mL) was added with stirring to a slurry of LiAlD₄ (Cambridge Isotopes, 98 atom % D, 1.63 g, 38.9 mmol, 1.5 equiv) in THF (6.4 ml) at −78° C. The reaction mixture was warmed to rt and stirred under N₂ for 2 h. The reaction was quenched by careful successive dropwise addition of 1.63 mL of water, 1.63 mL of 15% aqueous NaOH, and 4.89 mL of water. The inorganic precipitate was removed by filtration and the filtrate was concentrated in vacuo to 2.64 g (79%) of 20 as a clear oil.

Synthesis of 1-(Bromomethyl-d₂)-3-fluorobenzene (21). To a solution of alcohol 20 (2.04 g, 15.9 mmol) in CH₂Cl₂ (20 mL) at −20° C., was added dropwise with stirring PBr₃ (1.16 g, 28.7 mmol, 1.8 equiv). The reaction mixture was stirred for 3 h at −20° C., was warmed to 0° C. and stirred for 1 h, and then was quenched by the addition of water (24 mL). The mixture was pH-adjusted to pH 8 by the gradual addition of K₂CO₃. The layers were separated and the aqueous layer was extracted with CH₂Cl₂ (3×20 mL). The combined organic layers were dried over Na₂SO₄, filtered, and concentrated in vacuo to 1.8 g (59%) of 21 as a clear oil.

Synthesis of 4-((3-Fluorophenyl)methoxy-d₂)benzaldehyde (23). To a stirring solution of bromide 21 (0.500 g, 2.6 mmol) in DMF (20 mL) was added 4-hydroxybenzaldehyde 22 (0.352 g, 2.9 mmol, 1.1 equiv) followed by K₂CO₃ (0.742 g, 5.4 mmol, 2.1 equiv). The mixture was heated to 70° C. and stirred overnight under N₂. The resulting mixture was diluted with water (30 mL) and extracted with EtOAc (3×50 mL). The organic layer was dried over Na₂SO₄ and concentrated in vacuo to 556 mg (92%) of the product 23. HPLC (method: 150 mm C18-RP column-gradient method 5-95% ACN; Wavelength: 254 nm): retention time: 5.02 min. MS (M+H): 233.1.

Synthesis of (S)-2-(4-((3-Fluorophenyl)methoxy-d₂)benzylamino)-(3,3,3-d₃-propan)amide (108). To a stirring solution of aldehyde 23 (147 mg, 0.63 mmol) in MeOH (10 mL), was added 15 (89 mg, 0.70 mmol, 1.1 equiv) followed by triethylamine (100 μl, 0.72 mmol, 1.1 equiv). The mixture was heated to 40° C. and stirred for 1.5 h. Na(CN)BH₃ (44 mg, 0.70 mmol, 1.1 equiv) was added and the mixture was stirred for 5 days (d) at 40° C. with additional Na(CN)BH₃ (44 mg, 0.70 mmol, 1.1 equiv) added after each 1 d period. The mixture was cooled to rt, concentrated in vacuo, and the crude product was purified by automated flash column chromatography (90:10:1, CHCl₃:MeOH:NH₄OH) to yield 50.1 mg (21%) of pure product 108.

Subsequent formation of the HCl salt of 108 was achieved by stirring 108 (50.1 mg) in 1.25M HCl in ether for 30 min at rt. The resulting suspension was concentrated under reduced pressure, then dried under high vacuum to yield 43.3 mg of the HCl salt of 108.

The following analytical data is for 108 as the free base. ¹H-NMR (300 MHz, DMSO-d₆): δ 2.96 (s, 1H), 3.46 (d, J=13.1, 1H), 3.59 (d, J=113.1, 1H), 6.92-6.96 (m, 3H), 7.12-7.17 (m, 1H), 7.22-7.30 (m, 5H), 7.40-7.46 (m, 1H). HPLC (method: 150 mm C18-RP column-gradient method 5-95% ACN; Wavelength: 280 nm): retention time: 2.92 min; 93% purity. MS (M+H): 308.1.

### Example 5

Synthesis of (S)-2-(4-((3-Fluorophenyl)methoxy-d₂)benzylamino)-(2,3,3,3-d₄-propan)amide (105). Compound 105 was prepared as shown in Scheme 8 above using intermediate 12 in place of intermediate 15. Details of the synthesis are set forth below.

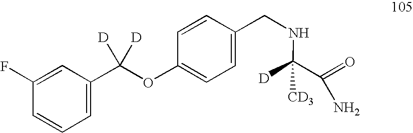

Synthesis of (S)-2-(4-((3-Fluorophenyl)methoxy-d₂)benzylamino)-(2,3,3,3-d₄-propan)amide (105). To a stirring solution of aldehyde 23 (100 mg, 0.43 mmol) in 1:1 MeOH/THF (10 mL), was added 12 (55 mg, 0.43 mmol, 1.0 equiv) followed by triethylamine (70 μl, 0.50 mmol, 1.2 equiv) and Na₂SO₄ (50 mg). The mixture was stirred at rt under N₂ for 1 h. Na(CN)BH₃ (162 mg, 2.6 mmol, 6.0 equiv) was added and the mixture was stirred for 3 d at rt. The resulting mixture was concentrated in vacuo, and the crude product was purified by automated flash column chromatography (0-10%, MeOH/CH₂Cl₂+0.1% NH₄OH) to yield 64 mg (43%) of pure product 105.

Subsequent formation of the HCl salt of 105 was achieved by stirring 105 (64 mg) in 1.25M HCl in ether for 30 min at rt. The resulting suspension was concentrated under reduced pressure, then dried under high vacuum to yield 59 mg of the HCl salt of 105.

The following analytical data is for 105 as the free base. ¹H-NMR (300 MHz, DMSO-d₆): δ 3.62 (d, J=13.1, 1H), 3.72 (d, J=13.4, 1H), 6.96-6.99 (m, 2H), 7.13-7.18 (m, 2H), 7.25-7.29 (m, 4H), 7.41-7.49 (m, 2H). HPLC (method: 150 mm C18-RP column-gradient method 5-95% ACN; Wavelength: 280 nm): retention time: 2.92 min; 95% purity. MS (M+H): 309.2.

### Example 6

Synthesis of (S)-2-((4-((3-Fluorophenyl)methoxy-d₂)phenyl)methyl-d₂-amino)-(2,3,3,3-d₄-propan)amide (100). Compound 100 was prepared as shown in Scheme 9 below. Details of the synthesis are set forth below.

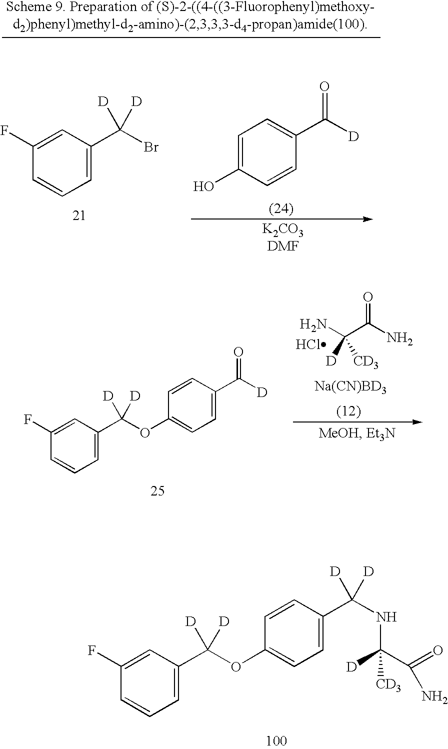

Synthesis of 4-((3-Fluorophenyl)methoxy-d₂)benzaldehyde-d₁ (25). To a stirring solution of the bromide 21 (0.500 g, 2.6 mmol) in DMF (20 mL) was added 4-hydroxybenzaldehyde-d₁ 24 (0.354 g, 2.9 mmol, 1.1 equiv) followed by K₂CO₃ (0.742 g, 5.4 mmol, 2.1 equiv). The mixture was heated to 70° C. and stirred overnight under N₂. The resulting mixture was diluted with water (30 mL) and extracted with EtOAc (3×50 mL). The organic layer was dried over Na₂SO₄ and concentrated in vacuo to 425 mg (70%) of the product 25. HPLC (method: 150 mm C18-RP column-gradient method 5-95% ACN; Wavelength: 254 nm): retention time: 4.99 min. MS (M+H): 234.1.

Synthesis of (S)-2-((4-((3-Fluorophenyl)methoxy-d₂)phenyl)methyl-d₂-amino)-(2,3,3,3-d₄-propan)amide (100). To a stirring solution of aldehyde 25 (100 mg, 0.43 mmol) in 1:1 MeOH/THF (10 mL), was added 12 (121 mg, 0.94 mmol, 1.1 equiv) followed by triethylamine (120 μl, 0.86 mmol, 1.0 equiv) and Na₂SO₄ (50 mg). The mixture was stirred at rt under N₂ for 1.5 h. The mixture was pH-adjusted to pH 4 using acetic acid-d₄. Na(CN)BD₃ (372 mg, 5.7 mmol, 6.6 equiv) was added and the mixture was heated to 50° C. and stirred overnight. The resulting mixture was concentrated in vacuo, and the crude product was purified by automated flash column chromatography (0-10%, MeOH/CH₂Cl₂+0.1% NH₄OH). The product 100 was then dissolved in MeOH (3 mL) followed by the addition of 1.25M HCl in ether (4 mL) and stirring was continued for 30 min at rt. The mixture was concentrated under reduced pressure then dried further under high vacuum to yield 86.5 mg (29%) of the HCl salt of 100. ¹H-NMR (300 MHz, DMSO-d₆): δ 7.06 (d, J=8.6, 2H), 7.14-7.18 (m, 1H), 7.26-7.29 (m, 2H), 7.42-7.44 (m, 3H), 7.61 (s, 1H), 7.968 (s, 1H), 9.04 (s, 1H), 9.42 (s, 1H). HPLC (method: 150 mm C18-RP column-gradient method 5-95% ACN; Wavelength: 280 nm): retention time: 2.82 min; 96% purity. MS (M+H of the free base): 311.2.

### Example 7

Synthesis of(S)-2-((4-((3-fluorophenyl)methoxy-d₂)phenyl)methyl-d₂-amino)-(3,3,3-d₃-propan)amide (101). Compound 101 was prepared according to Scheme 9 above using intermediate 15 in place of intermediate 12. Details of the synthesis are set forth below.

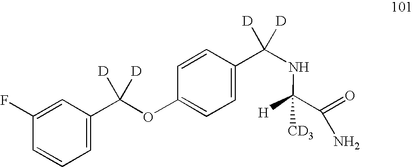

Synthesis of (S)-2-((4-((3-Fluorophenyl)methoxy-d₂)phenyl)methyl-d₂-amino)-(3,3,3-d₃-propan)amide (101). To a stirring solution of aldehyde 25 (100 mg, 0.43 mmol) in 1:1 MeOH/THF (10 mL), was added 15 (61 mg, 0.47 mmol, 1.1 equiv) followed by triethylamine (70 μl, 0.50 mmol, 1.2 equiv) and Na₂SO₄ (50 mg). The mixture was stirred at rt under N₂ for 1 h. Na(CN)BD₃ (170 mg, 2.6 mmol, 6.0 equiv) was added and the mixture was stirred for 1 day (d) at rt. Additional Na(CN)BD₃ (85 mg, 1.3 mmol, 3.0 equiv) was added and the mixture was stirred for 2 d at rt. The resulting mixture was concentrated in vacuo, and the crude product was purified by automated flash column chromatography (0-10%, MeOH/CH₂Cl₂+0.1% NH₄OH). The product 101 was then dissolved in MeOH (3 mL) followed by the addition of 1.25M HCl in ether (4 mL) and stirring was continued for 30 min at rt. The mixture was concentrated under reduced pressure then dried further under high vacuum to yield 20 mg (14%) of the HCl salt of 101. ¹H-NMR (300 MHz, DMSO-d₆): δ 3.69 (s, 1H), 7.06 (d, J=8.6, 2H), 7.14-7.18 (m, 1H), 7.26-7.29 (m, 2H), 7.39-7.45 (m, 3H), 7.62 (s, 1H), 7.96 (s, 1H), 9.03 (bs, 1H), 9.41 (bs, 1H). HPLC (method: 150 mm C18-RP column-gradient method 5-95% ACN; Wavelength: 360 nm): retention time: 2.98 min; 95% purity. MS (M+H of the free base): 310.1.

### Example 8

Synthesis of (S)-2-((4-((3-fluorophenyl)methoxy-d₂)phenyl-d₄)methyl-d₂-amino)-(2,3,3,3-d₄-propan)amide (109). Compound 109 was prepared as shown in Scheme 10 below. Details of the synthesis are set forth below.

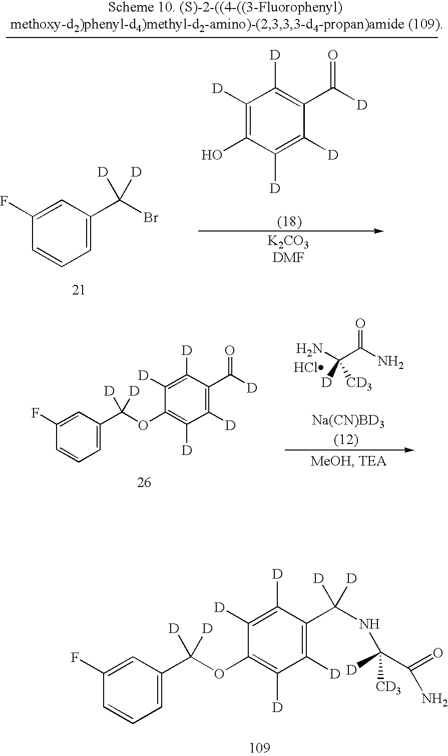

Synthesis of 4-((3-Fluorophenyl)methoxy-d₂)-2,3,5,6-d₄-benzaldehyde-d₁ (26). To a stirring solution of 21 (0.750 g, 3.9 mmol) in DMF (20 mL) was added 18 (0.549 g, 4.3 mmol, 1.1 equiv) followed by K₂CO₃ (1.112 g, 80.0 mmol, 2.1 equiv). The mixture was heated to 70° C. and stirred overnight under N₂. The resulting solution was allowed to cool to rt, diluted with D₂O (30 mL), and extracted with EtOAc (3×25 mL). The organic layer was dried over Na₂SO₄, filtered and concentrated in vacuo to yield 826 mg (89%) of the aldehyde 26. HPLC (method: 150 mm C18-RP column-gradient method 5-95% ACN; Wavelength: 254 nm): retention time: 5.00 min. MS (M+H): 238.1.

Synthesis of (S)-2-((4-((3-Fluorophenyl)methoxy-d₂)phenyl-d₄)methyl-d₂-amino)-(2,3,3,3-d₄-propan)amide (109). To a stirring solution of aldehyde 26 (100 mg, 0.43 mmol) in 1:1 MeOH/THF (10 mL), was added 12 (60 mg, 0.47 mmol, 1.1 equiv) followed by triethylamine (70 μl, 0.50 mmol, 1.2 equiv) and Na₂SO₄ (50 mg). The mixture was stirred at rt under N₂ for 1 h. Na(CN)BD₃ (167 mg, 2.6 mmol, 6.0 equiv) was added and the mixture was stirred for 3 d at rt. The resulting mixture was concentrated in vacuo, and the crude product was purified by automated flash column chromatography (0-10%, MeOH/CH₂Cl₂+0.1% NH₄OH). The product 109 was then dissolved in MeOH (3 mL) followed by the addition of 1.25M HCl in ether (4 mL) and stirring was continued for 30 min at rt. The mixture was concentrated under reduced pressure then dried further under high vacuum to yield 52 mg (34%) of the HCl salt of 109. ¹H-NMR (300 MHz, DMSO-d₆): δ 7.14-7.19 (m, 1H), 7.26-7.30 (m, 2H), 7.41-7.47 (m, 1H), 7.63 (s, 1H), 7.93 (s, 1H), 9.02 (bs, 1H), 9.22 (bs, 1H). HPLC (method: 150 mm C18-RP column-gradient method 5-95% ACN; Wavelength: 280 nm): retention time: 2.93 min; 95% purity. MS (M+H of the free base): 315.2.

### Example 9

Synthesis of (S)-2-((4-((3-fluorophenyl)methoxy-d₂)phenyl-d₄)methyl-d₂-amino)-(3,3,3-d₃-propan)amide (110). Compound 110 was prepared according to Scheme 10 above replacing intermediate 12 with intermediate 15. Details of the synthesis are set forth below.

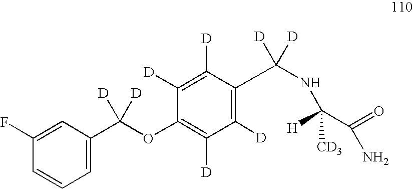

Synthesis of (S)-2-((4-((3-Fluorophenyl)methoxy-d₂)phenyl-d₄)methyl-d₂-amino)-(3,3,3-d₃-propan)amide (110). To a stirring solution of aldehyde 26 (100 mg, 0.43 mmol) in 1:1 MeOH/THF (10 mL), was added 15 (59 mg, 0.46 mmol, 1.1 equiv) followed by triethylamine (70 μl, 0.50 mmol, 1.2 equiv) and Na₂SO₄ (50 mg). The mixture was stirred at rt under N₂ for 1 h. Na(CN)BD₃ (167 mg, 2.6 mmol, 6.0 equiv) was added and the mixture was stirred for 3 d at rt. The resulting mixture was concentrated in vacuo, and the crude product was purified by automated flash column chromatography (0-10%, MeOH/CH₂Cl₂+0.1% NH₄OH) to yield 97 mg (72%) of pure product 110.

Subsequent formation of the HCl salt of 110 was achieved by stirring 110 (97 mg) in 1.25M HCl in ether for 30 min at rt. The resulting suspension was concentrated under reduced pressure, then dried under high vacuum to yield 76 mg (95% pure) of the HCl salt of 110.

The following analytical data is for 110 as the free base. ¹H-NMR (300 MHz, DMSO-d₆): δ 3.25 (s, 1H), 7.13-7.18 (m, 1H), 7.24-7.29 (m, 3H), 7.41-7.46 (m, 1H), 7.54 (bs, 1H). HPLC (method: 150 mm C18-RP column-gradient method 5-95% ACN; Wavelength: 280 nm): retention time: 2.93 min; 95% purity. MS (M+H): 314.2.

Without further description, it is believed that one of ordinary skill in the art can, using the preceding description and the illustrative examples, make and utilize the compounds of the present invention and practice the claimed methods. It should be understood that the foregoing discussion and examples merely present a detailed description of certain preferred embodiments. It will be apparent to those of ordinary skill in the art that various modifications and equivalents can be made without departing from the spirit and scope of the invention. All the patents, journal articles and other documents discussed or cited above are herein incorporated by reference.

1. A compound of the formula A 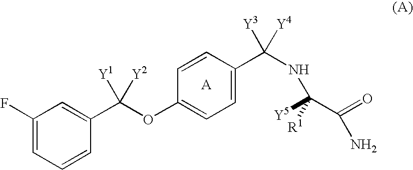
or a pharmaceutically acceptable salt thereof, wherein:
Ring A contains 0-4 deuterium atoms;
each Y is independently selected from hydrogen and deuterium;
R¹ is selected from —CH₃, —CH₂D, —CHD₂ and —CD₃; and
when R¹ is —CH₃ and Ring A contains 0 deuterium atoms, then at least one Y is deuterium.

2. The compound of claim 1, wherein:
Y¹ and Y² are the same;
Y³ and Y⁴ are the same;
Ring A contains 0 or 4 deuterium atoms; and
R¹ is selected from —CH₃ and —CD₃.

3. A compound of formula I 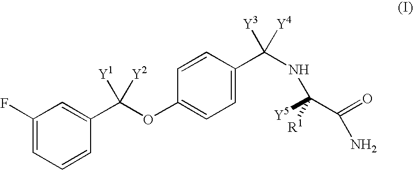
or a pharmaceutically acceptable salt, solvate, or hydrate thereof, wherein:
each Y is independently selected from hydrogen and deuterium;
R¹ is selected from —CH₃, —CH₂D, —CHD₂ and —CD₃; and
when R¹ is —CH₃, at least one Y is deuterium.

4. The compound of claim 3, wherein:
Y¹ and Y² are the same;
Y³ and Y⁴ are the same; and
R¹ is selected from —CH₃ and —CD₃.

5. The compound of claim 1, selected from any one of the following compounds: 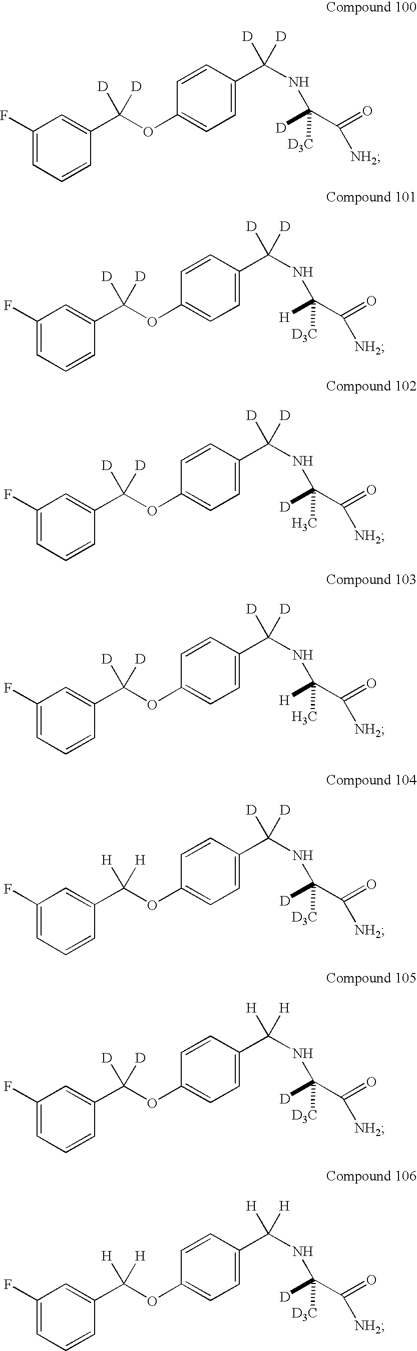 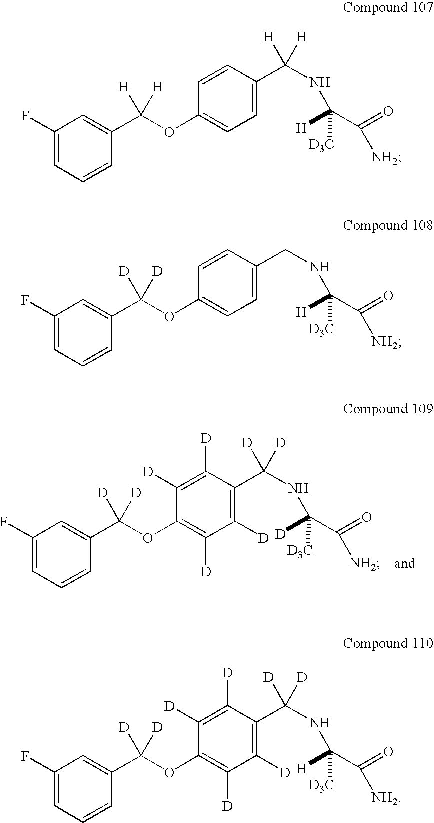
or a pharmaceutically acceptable salt of any of the foregoing.

6. A pyrogen-free pharmaceutical composition comprising a compound of formula A 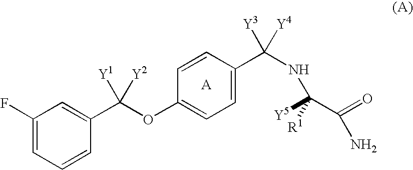
or a pharmaceutically acceptable salt thereof, wherein:
Ring A contains 0-4 deuterium atoms;
each Y is independently selected from hydrogen and deuterium;
R¹ is selected from —CH₃, —CH₂D, —CHD₂ and —CD₃; and
when R¹ is —CH₃ and Ring A contains 0 deuterium atoms, then at least one Y is deuterium; and
a pharmaceutically acceptable carrier.

7. The composition of claim 6, further comprising a second therapeutic agent useful in the treatment of a disease or condition selected from Parkinson's disease, restless legs syndrome, addictive disorders, head pain conditions, chronic pain, neuropathic pain, epilepsy, and depression.

8. The composition of claim 7, wherein the second therapeutic agent is selected from an antispastic agent and a hypnotic agent.

9. A method of modulating MAO-B, Na⁺ channel, and/or Ca²⁺ channel activity in a cell, comprising contacting the cell with a compound of formula A 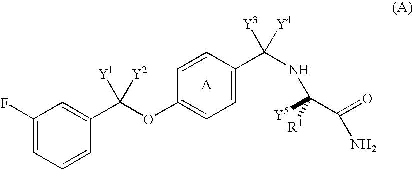
or a pharmaceutically acceptable salt thereof, wherein:
Ring A contains 0-4 deuterium atoms;
each Y is independently selected from hydrogen and deuterium;
R¹ is selected from —CH₃, —CH₂D, —CHD₂ and —CD₃; and
when R¹ is —CH₃ and Ring A contains 0 deuterium atoms then at least one Y is deuterium.

10. A method of treating a disease or condition selected from Parkinson's disease, restless legs syndrome, addictive disorders, head pain conditions, chronic pain, neuropathic pain, epilepsy, and depression, in a patient in need thereof comprising the step of administering to the patient an effective amount of a composition comprising a compound of formula A 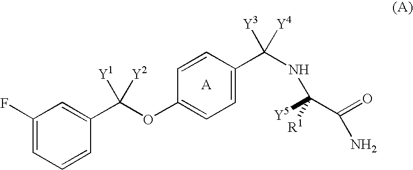
or a pharmaceutically acceptable salt thereof, wherein:
Ring A contains 0-4 deuterium atoms;
each Y is independently selected from hydrogen and deuterium;
R¹ is selected from —CH₃, —CH₂D, —CHD₂ and —CD₃; and
when R¹ is —CH₃ and Ring A contains 0 deuterium atoms then at least one Y is deuterium; and
a pharmaceutically acceptable carrier.

11. The method of claim 10, wherein the disease or condition is selected from Parkinson's disease, and restless legs syndrome.

12. The method of claim 10 comprising the additional step of co-administering to the patient a second therapeutic agent useful in the treatment of a disease or condition selected from Parkinson's disease, restless legs syndrome, addictive disorders, head pain conditions, chronic pain, neuropathic pain, epilepsy, and depression.

13. The method of claim 12, wherein the second therapeutic agent is selected from an antispastic agent and a hypnotic agent.

14. A pyrogen-free pharmaceutical composition comprising a compound of formula I 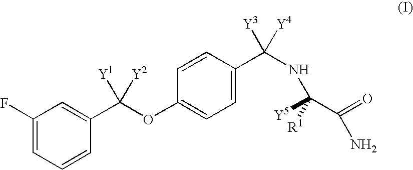
or a pharmaceutically acceptable salt, solvate, or hydrate thereof, wherein:
each Y is independently selected from hydrogen and deuterium;
R¹ is selected from —CH₃, —CH₂D, —CHD₂ and —CD₃; and
when R¹ is —CH₃, at least one Y is deuterium; and
a pharmaceutically acceptable carrier.

15. The composition of claim 14, further comprising a second therapeutic agent useful in the treatment of a disease or condition selected from Parkinson's disease, restless legs syndrome, addictive disorders, head pain conditions, chronic pain, neuropathic pain, epilepsy, and depression.

16. A method of modulating MAO-B, Na⁺ channel, and/or Ca²⁺ channel activity in a cell, comprising contacting the cell with a compound of formula I 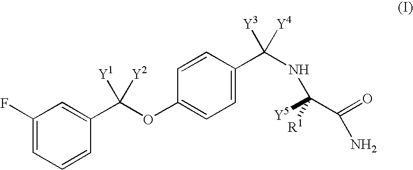
or a pharmaceutically acceptable salt, solvate, or hydrate thereof, wherein:
each Y is independently selected from hydrogen and deuterium;
R¹ is selected from —CH₃, —CH₂D, —CHD₂ and —CD₃; and
when R¹ is —CH₃, at least one Y is deuterium.

17. A method of treating a disease or condition selected from Parkinson's disease, restless legs syndrome, addictive disorders, head pain conditions, chronic pain, neuropathic pain, epilepsy, and depression, in a patient in need thereof comprising the step of administering to the patient an effective amount of a composition comprising a compound of formula I 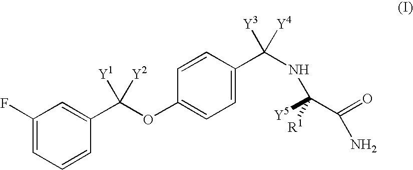
or a pharmaceutically acceptable salt, solvate, or hydrate thereof, wherein:
each Y is independently selected from hydrogen and deuterium;
R¹ is selected from —CH₃, —CH₂D, —CHD₂ and —CD₃; and
when R¹ is —CH₃, at least one Y is deuterium; and
a pharmaceutically acceptable carrier.

18. The method of claim 17, wherein the disease or condition is selected from Parkinson's disease, and restless legs syndrome.

19. The method of claim 17 comprising the additional step of co-administering to the patient a second therapeutic agent useful in the treatment of a disease or condition selected from Parkinson's disease, restless legs syndrome, addictive disorders, head pain conditions, chronic pain, neuropathic pain, epilepsy, and depression.

20. The method of claim 19, wherein the second therapeutic agent is selected from an antispastic agent and a hypnotic agent.
